(19) United States
(12) Patent Application Publication
(10) Pub. No.: US 2007/0133737 A1
(43) Pub. Date: Jun. 14, 2007

(54) COLLIMATOR UNIT, REINFORCED COLLIMATOR, DETECTOR FOR CT, AND CT SYSTEM

(76) Inventors: Mitsuru Yahata, Tokyo (JP); Katsumasa Nose, Tokyo (JP)

Correspondence Address: Patrick W. Rasche;Armstrong Teasdale LLP, Suite 2600, One Metropolitan Square, St. Louis, MO, 63102, US

(21) Appl. No.: 11/567,813

(22) Filed: Dec. 7, 2006

(30) Foreign Application Priority Data
Dec. 8, 2005 (JP) 2005-354451

(51) Int. Cl. H05G 1/60; A61B 6/00; G01N 23/00; G21K 1/12; G21K 1/02

(52) U.S. Cl. 378/19; 378/147

(57) ABSTRACT

To reduce a collimator thickness, plural collimators are not erected each independently on a scintillator array but are integrated into a collimator unit by molding with use of a plastic resin and the collimator unit is installed on the scintillator array. Even if the collimator thickness is made small, resulting in deterioration of the rigidity strength, there occurs no problem and thus the collimator thickness can be made as small as 200 μm or less.

### CROSS REFERENCE TO RELATED APPLICATIONS

This application claims the benefit of Japanese Application No. 2005-354451 filed Dec. 8, 2005.

### BACKGROUND OF THE INVENTION

The present invention relates to a collimator unit, a reinforced collimator, a detector for CT (Computed Tomography), and a CT system. More particularly, the present invention is concerned with a collimator unit which permits reduction of the collimator thickness, a reinforced collimator, a detector for CT of a high geometric efficiency using the collimator unit or the reinforced collimator, and a CT system of a high resolution using the detector for CT.

In a conventional X-ray detector, a collimator is erected between scintillator channels for the purpose of removing scattered lines (see, for example, Japanese Patent Laid-Open Publication No. 2004-93489).

Recently, setting the detector pitch at 1 mm or smaller has been conducted for the improvement of resolution.

However, even if an attempt is made to reduce the collimator thickness (e.g., 200 μm or smaller), the rigidity strength is deteriorated and it becomes difficult to erect a collimator between scintillator channels, thus giving rise to the problem that the collimator thickness cannot be made smaller.

Accordingly, it is an object of the present invention to provide a collimator unit permitting reduction of the collimator thickness, a reinforced collimator, a detector for CT of a high geometric efficiency using the collimator unit or the reinforced collimator, and a CT system using the detector for CT.

### SUMMARY OF THE INVENTION

In a first aspect of the present invention there is provided a collimator unit comprising a plurality of collimators made integral using a material lower in X-ray absorption coefficient than the collimators.

According to the collimator unit in the above first aspect, since plural collimators are not erected each individually but are made integral using a material lower in X-ray absorption coefficient than the collimators, even if the collimator thickness is made small with consequent deterioration of the rigidity strength, there occurs no problem. Thus, the collimator thickness can be made small.

The material lower in X-ray absorption coefficient is, for example, a plastic resin or a carbon fiber.

In a second aspect of the present invention there is provided, in combination with the above first aspect, a collimator unit wherein the material lower in X-ray absorption coefficient is a plastic resin.

According to the collimator unit in the above second aspect, since a plastic resin is used for making the plural collimators integral, the collimator unit can be manufactured less expensively.

In a third aspect of the present invention there is provided, in combination with the above second aspect, a collimator unit wherein the plural collimators are made integral by molding with use of the plastic resin.

According to the collimator unit in the above third aspect, since the plural collimators are molded using the plastic resin, the collimator unit is suitable for mass production.

In a fourth aspect of the present invention there is provided a reinforced collimator comprising a collimator reinforced using a material lower in X-ray absorption coefficient than the collimator.

According to the reinforced collimator in the above fourth aspect, since the collimator is reinforced using a material lower in X-ray absorption coefficient than the collimator, it is possible to ensure a required rigidity strength as a whole even if the collimator thickness is made small. Thus, the collimator thickness can be made small.

The material lower in X-ray absorption coefficient is, for example, a plastic resin or a carbon fiber.

In a fifth aspect of the present invention there is provided, in combination with the above fourth aspect, a reinforced collimator wherein the material lower in X-ray absorption coefficient is a carbon fiber.

According to the reinforced collimator in the above fifth aspect, since a carbon fiber is used for reinforcing the collimator, it is possible to reinforce the collimator with a sufficient strength even if the reinforcing thickness is small.

In a sixth aspect of the present invention there is provided, in combination with the above fifth aspect, a reinforced collimator wherein thin sheets of the carbon fiber are laminated to both surfaces of the collimator.

According to the reinforced collimator in the above sixth aspect, since thin sheets of the carbon fiber are laminated to both surfaces of the collimator, not only a sufficient strength is obtained but also X-ray absorption can be minimized.

In a seventh aspect of the present invention there is provided a detector for CT, including a collimator unit comprising a plurality of collimators made integral using a material lower in X-ray absorption coefficient than the collimators.

According to the detector for CT in the above seventh aspect, both permitting reduction of the collimator thickness, a high geometric efficiency can be attained even if the detector pitch in the channel direction is made small.

In an eighth aspect of the present invention there is provided a detector for CT, including a reinforced collimator comprising a collimator reinforced using a material lower in X-ray absorption coefficient than the collimator.

According to the detector for CT in the above eighth aspect, both permitting reduction of the collimator thickness, a high geometric efficiency can be attained even if the detector pitch in the channel direction is made small.

In a ninth aspect of the present invention there is provided a CT system including a X-ray detector and a X-ray generator, wherein said X-ray detector including a collimator unit comprising a plurality of collimators made integral using a material lower in X-ray absorption coefficient than the collimators.

According to the CT system in the above ninth aspect, since there is used the detector for CT permits reduction of the detector pitch in the channel direction, it is possible to attain a high resolution.

In a tenth aspect of the present invention there is provided a CT system including a X-ray detector and a X-ray generator, wherein said X-ray detector including a reinforced collimator comprising a collimator reinforced using a material lower in X-ray absorption coefficient than the collimator.

According to the CT system in the above tenth aspect, since there is used the detector for CT permits reduction of the detector pitch in the channel direction, it is possible to attain a high resolution.

According to the collimator unit and reinforced collimator of the present invention it is possible to make the collimator thickness small.

According to the detector for CT of the present invention it is possible to attain a high geometric efficiency even if the detector pitch in the channel direction is made small.

According to the CT system of the present invention it is possible to attain a high resolution.

### BRIEF DESCRIPTION OF THE DRAWINGS

FIG. 1 is a block diagram showing the construction of an X-ray CT system according to a first embodiment of the present invention;

FIG. 2 is a longitudinal sectional view showing an X-ray detector according to the first embodiment;

FIG. 3 is a top view thereof;

FIG. 4 is a longitudinal sectional view showing an X-ray detector according to a second embodiment of the present invention; and

FIG. 5 is a longitudinal sectional view showing an X-ray detector according to a third embodiment of the present invention.

### DETAILED DESCRIPTION OF THE INVENTION

The present invention will be described in more detail hereinunder by way of embodiments thereof illustrated in the drawings. The present invention is not limited by the following embodiments.

### First Embodiment

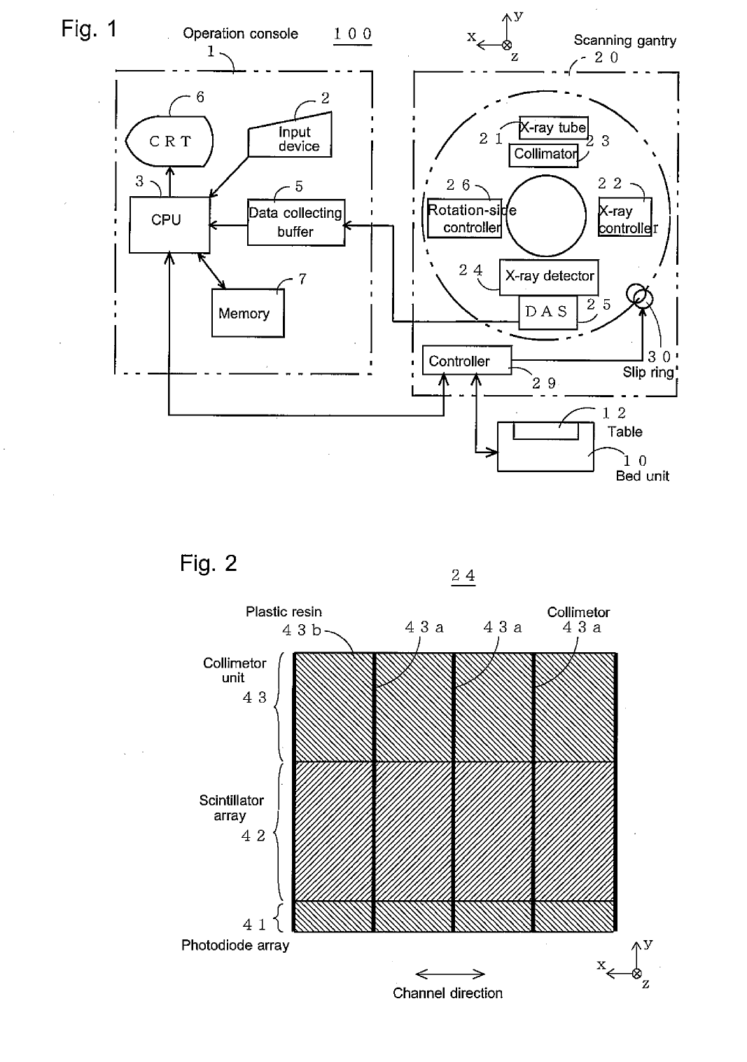

FIG. 1 is a block diagram showing the construction of an X-ray CT system 100 according to a first embodiment of the present invention.

The X-ray CT system 100 includes an operation console 1, a bed unit 10, and a scanning gantry 20.

The operation console 1 includes an input device 2 which receives an input from an operator, a central processing unit 3 which executes for example a scan processing and an image reconstruction processing, a data collecting buffer 5 for collecting projection data acquired by the scanning gantry 20, a CRT 6 which displays a generated CT image, and a memory 7 for the storage of programs, data and CT image.

The bed unit 10 includes a table 12, the table 12 being adapted to move into and out of a bore (hollow portion) of the scanning gantry 20 while carrying thereon an object to be radiographed. The table 12 is moved vertically and horizontally by means of a motor installed within the bed unit 10.

The scanning gantry 20 includes an X-ray tube 21, an X-ray controller 22, a collimator 23, an X-ray detector 24, a DAS (Data Acquisition System) 25, a rotation-side controller 26 for controlling the X-ray controller 22, collimator 23 and DAS 25, a controller 29 which provides and receives controls signals, etc. to and from the operation console 1 and the bed unit 10, and a slip ring 30.

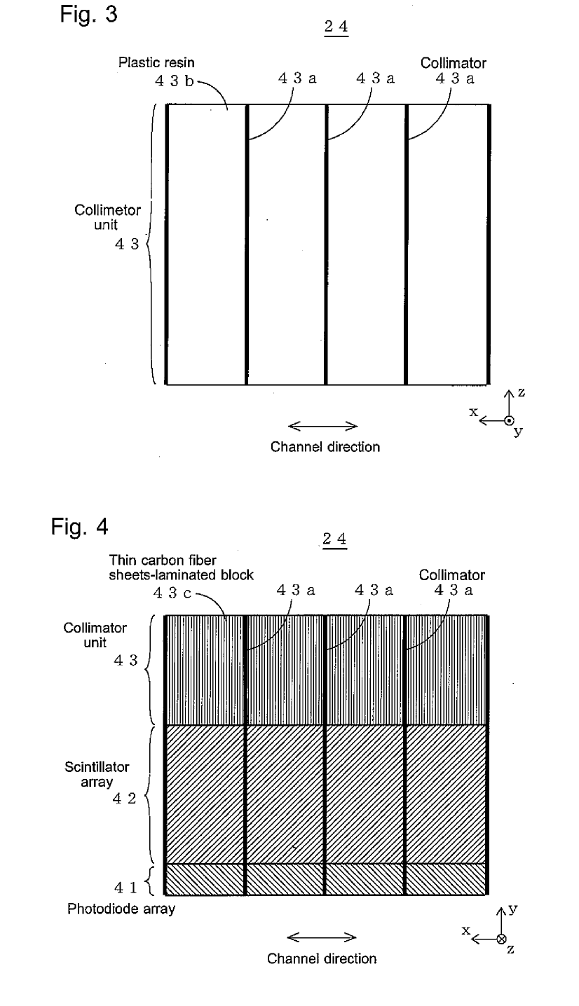

FIG. 2 is a longitudinal sectional view showing the X-ray detector 24 and FIG. 3 is a top view thereof.

The X-ray detector 24 includes a photodiode array 41 of photodiodes of plural channels, a scintillator array 42 of scintillators of plural channels, and a collimator unit 43 installed on an upper surface of the scintillator array 42.

The collimator unit 43 is made up of plural collimators 43a which provide separation between channels and a plastic resin 43b which molds the collimators 43a integrally.

The collimators 43a are made of tungsten or lead and are each 200 μm or less in thickness.

The plastic resin 43b is a thermoplastic resin such as polyethylene or polyvinyl chloride and the thickness thereof, i.e., the spacing between adjacent collimators 43a, i.e., the detector pitch in the channel direction, is 1 mm or less.

In the collimator unit 43 according to this first embodiment, the plural collimators 43a are not erected each independently but are molded integrally by the plastic resin 43b. Therefore, even if the collimators 43a are made small in thickness, resulting in deterioration of the rigidity strength, there occurs no problem. Thus, the thickness of each collimator 43a can be made as small as 200 μm or less.

Moreover, since the detector 24 for CT according to this first embodiment uses the collimator unit 43 which permits reduction in thickness of the collimator 43a, it is possible to attain a high geometric efficiency even if the detector pitch in the channel direction is made small.

Further, since the X-ray CT system 100 according to this first embodiment uses the X-ray detector 24 which permits reduction of the detector pitch in the channel direction, it is possible to attain a high resolution.

### Second Embodiment

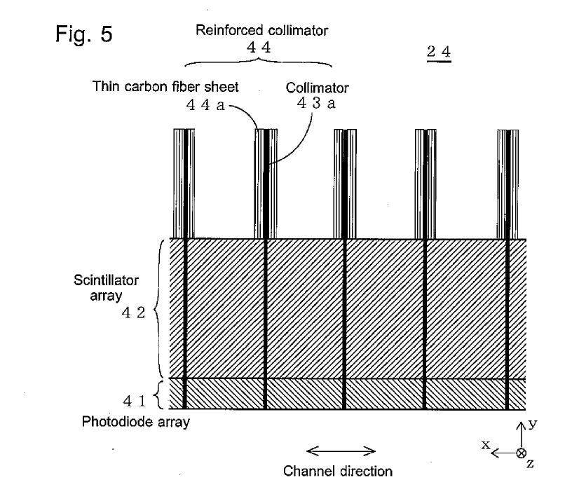

FIG. 4 is a longitudinal sectional view showing an X-ray detector 24 according to a second embodiment of the present invention.

The X-ray detector 24 includes a photodiode array 41 of photodiodes of plural channels, a scintillator array 42 of scintillators of plural channels, and a collimator unit 43 installed on an upper surface of t he scintillator array 42.

The collimator unit 43 is made up of plural collimators 43a which provide separation between channels and thin carbon fiber sheets-laminated blocks 43c each sandwiched between adjacent collimators 43a, the collimators 43a and the blocks 43c being made integral by bonding.

The collimators 43a are each made of tungsten or lead and are each 200 μm or less in thickness.

The thin carbon fiber sheets-laminated blocks 43c are each formed by laminating a large number of thin carbon fiber sheets each 100 μm or so in thickness into a block. The thickness of each block 43c, i.e., the spacing between adjacent collimators 43a, i.e., the detector pitch in the channel direction, is 1 mm or less.

In the collimator unit 43 according to this second embodiment, the plural collimators 43a are not erected each independently but are made integral by bonding through the thin carbon fiber sheets-laminated blocks 43c. Therefore, no problem occurs even if the rigidity strength is deteriorated as a result of reduction in thickness of the collimators 43a, thus making it possible to reduce the thickness of each collimator 43a to 200 μm or less.

### Third Embodiment

FIG. 5 is a longitudinal sectional view showing an X-ray detector 24 according to a third embodiment of the present invention.

The X-ray detector 24 includes a photodiode array 41 of photodiodes of plural channels, a scintillator array 42 of scintillators of plural channels, and reinforced collimators 44 installed on an upper surface of the scintillator array 42.

The reinforced collimators 44 are each made up of a collimator 43a which provides separation between channels and thin carbon fiber sheets 44a disposed in a sandwiching relation to the collimator 43a, the collimator 43a and the thin carbon fiber sheets 44a being made integral by bonding.

The collimator 43a is made of tungsten or lead and is 200 μm or less in thickness.

The thin carbon fiber sheets 44a are a laminate of several thin carbon sheets each 100 μm or so in thickness.

In each reinforced collimator 44 according to this third embodiment, since the thin carbon fiber sheets 44a are bonded to both surfaces of the collimator 43a, it is possible to ensure a required rigidity strength even if the thickness of the collimator 43a is made small. Consequently, the thickness of the collimator 43a can be made as small as 200 μm or less. Besides, since the scintillators are not covered from above with the thin carbon fiber sheets, X-ray absorption can be minimized.

### INDUSTRIAL APPLICABILITY

The collimator unit, reinforced collimator, detector for CT and CT system according to the present invention are applicable to tomography.

1. A collimator unit comprising a plurality of collimators made integral using a material lower in X-ray absorption coefficient than the collimators.

2. A collimator unit according to claim 1, wherein the material lower in X-ray absorption coefficient is a plastic resin.

3. A collimator unit according to claim 2, wherein the plural collimators are made integral by molding with use of the plastic resin.

4. A reinforced collimator comprising a collimator reinforced using a material lower in X-ray absorption coefficient than the collimator.

5. A reinforced collimator according to claim 4, wherein the material lower in X-ray absorption coefficient is a carbon fiber.

6. A reinforced collimator according to claim 5, wherein thin sheets of the carbon fiber are laminated to both surfaces of the collimator.

7. A detector for CT,
including a collimator unit comprising a plurality of collimators made integral using a material lower in X-ray absorption coefficient than the collimators.

8. A detector for CT,
including a reinforced collimator comprising a collimator reinforced using a material lower in X-ray absorption coefficient than the collimator.

9. A CT system including a X-ray detector and a X-ray generator, wherein said X-ray detector including a collimator unit comprising a plurality of collimators made integral using a material lower in X-ray absorption coefficient than the collimators.

10. A CT system including a X-ray detector and a X-ray generator, wherein said X-ray detector including a reinforced collimator comprising a collimator reinforced using a material lower in X-ray absorption coefficient than the collimator.
